SEQUENCE LISTING (6 sequences)

SEQ ID NO: 1 - DNA, 1780 bp, romaine lettuce
  n is an unspecified nucleotide
gaaacaaatgacgtgaaagttcttcaaaattgaattaattgtaatcctgaaaacttgatttgtgatagaagaatcaatggagtgctttggagctcgaaacatgacggcaacaatggcggtttttacgtgccctagattcacggactgtaatatcaggcacaaattttcgttactgaaacaacgaagatttactaatttatcagcatcgtcttcgttgcgtcaaattaagtgcagcgctaaaagcgaccgttgtgtagtggataaacaagggatttccgtagcagacgaagaagattatgtgaaggccggtggatcggagctgttttttgttcaaatgcagcggactaagtccatggaaagccagtctaaactttccgaaaagctagcacagataccaattggaaattgcatacttgatctggttgtaatcggttgtggccctgctggccttgctcttgctgcagagtcagccaaactagggttgaacgttggactcattggccctgatcttccttttacaaacaattatggtgtttggcaggatgaatttataggtcttggacttgaaggatgcattgaacattcttggaaagatactcttgtataccttgatgatgctgatcccatccgcataggtcgtgcatatggcagagttcatcgtgatttacttcatgaagagttgttaagaaggtgtgtggaatcaggtgtttcatatctaagctccaaagtagaaagaatcactgaagctccaaatggctatagtctcattgaatgtgaaggcaatatcaccattccatgcaggcttgctactgttgcatcaggggcagcttcagggaaatttctggagtatgaacttgggggtccccgtgtttgtgtccaaacagcttatggtatagaggttgaggttgaaaacaacccctatgatccagatctaatggtgttcatggattatagagacttctcaaaacataaaccggaatctttagaagcaaaatatccgactttcctctatgtcatggccatgtctccaacaaaaatattcttcgaggaaacttgtttagcttcaagagaagccatgcctttcaatcttctaaagtccaaactcatgtcacgattaaaggcaatgggtatccgaataacaagaacgtacgaagaggaatggtcgtatatccccgtaggtggatcgttacctaatacagaacaaaagaatctcgcatttggtgctgcagctagtatggtgcaccctgccacagggtattcagttgttcgatctttgtcagaagctcctaattatgcagcagtcattgctaagattttaagacaagatcaatctaaagagatgatttctcttggaaaatacactaacatttcaaaacaagcatgggaaacattgtggccacttgaaaggaaaagacagcgagccttctttctattcggactatcacacatcgtgctaatngatctagagggaacacgtacatttttccgtactttctttcgtttgcccaaatggatgtggtggggatttttggggtcttctttatcttcaacggatttgataatatttgcgctttatatgtttgtgatagcacctcacagcttgagaatggaactggttagacatctactttctgatccgacaggggcaactatggtaaaagcatatctcactatatagatttagattatataaataatacccatatcttgcatatatataagccttatttatttcttttgtacccccacaacaacatactcgttaattatatgtttttta

SEQ ID NO: 2 - protein, 533 aa, romaine lettuce
MECFGARNMTATMAVFTCPRFTDCNIRHKFSLLKQRRFTNLSASSSLRQIKCSAKSDRCVVDKQGISVADEEDYVKAGGSELFFVQMQRTKSMESQSKLSEKLAQIPIGNCILDLVVIGCGPAGLALAAESAKLGLNVGLIGPDLPFTNNYGVWQDEFIGLGLEGCIEHSWKDTLVYLDDADPIRIGRAYGRVHRDLLHEELLRRCVESGVSYLSSKVERITEAPNGYSLIECEGNITIPCRLATVASGAASGKFLEYELGGPRVCVQTAYGIEVEVENNPYDPDLMVFMDYRDFSKHKPESLEAKYPTFLYVMAMSPTKIFFEETCLASREAMPFNLLKSKLMSRLKAMGIRITRTYEEEWSYIPVGGSLPNTEQKNLAFGAAASMVHPATGYSVVRSLSEAPNYAAVIAKILRQDQSKEMISLGKYTNISKQAWETLWPLERKRQRAFFLFGLSHIVLMDLEGTRTFFRTFFRLPKWMWWGFLGSSLSSTDLIIFALYMFVIAPHSLRMELVRHLLSDPTGATMVKAYLTI

SEQ ID NO: 3 - protein, 524 aa, Arabidopsis
MECVGARNFAAMAVSTFPSWSCRRKFPVVKRYSYRNIRFGLCSVRASGGGSSGSESCVAVREDFADEEDFVKAGGSEILFVQMQQNKDMDEQSKLVDKLPPISIGDGALDHVVIGCGPAGLALAAESAKLGLKVGLIGPDLPFTNNYGVWEDEFNDLGLQKCIEHVWRETIVYLDDDKPITIGRAYGRVSRRLLHEELLRRCVESGVSYLSSKVDSITEASDGLRLVACDDNNVIPCRLATVASGAASGKLLQYEVGGPRVCVQTAYGVEVEVENSPYDPDQMVFMDYRDYTNEKVRSLEAEYPTFLYAMPMTKSRLFFEETCLASKDVMPFDLLKTKLMLRLSTLGIRILKTYEEEWSYIPVGGSLPNTEQKNLAFGAAASMVHPATGYSVVRSLSEAPKYASVIAEILREETTKQINSNISRQAWDTLWPPERKRQRAFFLFGLALIVQFDTEGIRSFFRTFFRLPKWMWQGFLGSTLTSGDLVLFALYMFVISPNNLRKGLINHLISDPTGATMIKTYLKV

SEQ ID NO: 4 - DNA, 1848 bp, Adonis palaestina
gagagaaaaagagtgttatattaatgttactgtcgcattcttgcaacacatattcagactccattttcttgttttctcttcaaaacaacaaactaatgtgacggagtatctagctatggaactacttggtgttcgcaacctcatctcttcttgccctgtctggacttttggaacaagaaaccttagtagttcaaaactagcttataacatacatcgatatggttcttcttgtagagtagattttcaagtgagggctgatggtggaagcgggagtagaacttctgttgcttataaagagggttttgtggacgaggaggattttatcaaagctggtggttctgagcttttgtttgtccaaatgcagcaaacaaagtctatggagaaacaggccaagctcgccgataagttgccaccaatacctttcggagaatctgtgatggacttggttgtaataggttgtggacctgctggtctttcactggctgcagaagctgctaagctaggcttgaaagttggccttattggtcctgatcttccttttacaaataattatggtgtgtgggaagacgagttcaaagatcttggacttgaacgttgtatcgagcatgcttggaaggacaccatcgtatatcttgacaatgatgctcctgtccttattggtcgtgcatatggacgagttagccggcatttgctgcatgaagagttgctgaaaaggtgtgtcgagtcaggtgtatcatatctgaattctaaagtggaaaggatcactgaagctggtgatggccatagtcttgtagtttgtgaaaacgacatctttatcccttgcaggcttgctactgttgcatctggagcagcttcagggaaacttttggagtatgaagtaggtggccctcgtgtttgtgtccaaactgcttatggtgtggaggttgaggtggagaacaatccatacgatcccaacttaatggtatttatggactacagagactatatgcaacagaaattacagtgctcggaagaagaatatccaacatttctctatgtcatgcccatgtcgccaacaagacttttttttgaggaaacctgtttggcctcaaaagatgccatgcctttcgatctactgaagagaaaactaatgtcacgattgaagactctgggtatccaagttacaaaaatttatgaagaggaatggtcttatattcctgttgggggttctttaccaaacacagagcaaaagaacctagcatttggtgctgcagcaagcatggtgcatccagcaacaggctattcggttgtacgatcactatcagaagctccaaaatatgcttctgtaattgcaaagattttgaagcaagataactctgcatatgtggtttctggacaaagcagtgcagtaaacatttcaatgcaagcatggagcagtctttggccaaaggagcgaaaacgtcaaagagcattctttcttttcgggttagagcttattgtgcagctagatattgaagcaaccagaacgttctttagaaccttcttccgcttgccaacttggatgtggtggggtttccttgggtcttcactatcatctttcgatcttgtattgttttccatgtacatgtttgttttggccccgaacagcatgaggatgtcacttgtgagacatttgctttcagatccttctggtgcagttatggttaaagcttacctcgaaaggtaatctgttttatgaaactatagtgtctcattaaataaatgaggatccttcgtatatgtatatgatcatctctatgtatatcctatattctaatctcataaagtaatcgaaaattcattgatagaaaaaaaaaaaaaaaaaaaaaa

SEQ ID NO: 5 - protein, 529 aa, Adonis palaestina
MELLGVRNLISSCPVWTFGTRNLSSSKLAYNIHRYGSSCRVDFQVRADGGSGSRTSVAYKEGFVDEEDFIKAGGSELLFVQMQQTKSMEKQAKLADKLPPIPFGESVMDLVVIGCGPAGLSLAAEAAKLGLKVGLIGPDLPFTNNYGVWEDEFKDLGLERCIEHAWKDTIVYLDNDAPVLIGRAYGRVSRHLLHEELLKRCVESGVSYLNSKVERITEAGDGHSLVVCENDIFIPCRLATVASGAASGKLLEYEVGGPRVCVQTAYGVEVEVENNPYDPNLMVFMDYRDYMQQKLQCSEEEYPTFLYVMPMSPTRLFFEETCLASKDAMPFDLLKRKLMSRLKTLGIQVTKIYEEEWSYIPVGGSLPNTEQKNLAFGAAASMVHPATGYSVVRSLSEAPKYASVIAKILKQDNSAYVVSGQSSAVNISMQAWSSLWPKERKRQRAFFLFGLELIVQLDIEATRTFFRTFFRLPTWMWWGFLGSSLSSFDLVLFSMYMFVLAPNSMRMSLVRHLLSDPSGAVMVKAYLER

SEQ ID NO: 6 - protein, 530 aa, Adonis palaestina
MELLGVRNLISSCPVWTFGTRNLSSSKLAYNIHRYGSSCRVDFQVRADGGSGSRSSVAYKEGFVDEEDFIKAGGSELLFVQMQQTKSMEKQAKLADKLPPIPFGESVMDLVVIGCGPAGLSLAAEAAKLGLKVGLIGPDLPFTNNYGVWEDEFKDLGLERCIEHAWKDTIVYLDNDAPVLIGRAYGRVSRHLLHEELLKRCVESGVSYLDSKVERITEAGDGHSLVVCENEIFIPCRLATVASGAASGKLLEYEVGGPRVCVQTAYGVEVEVENNPYDPNLMVFMDYRDYMQQKLQCSEEEYPTFLYVMPMSPTRLFFEETCLASKDAMPFDLLKRKLMSRLKTLGIQVTKVYEEEWSYIPVGGSLPNTEQKNLAFGAAASMVHPATGYSVVRSLSEAPKYASVIAKILKQDNSAYVVSGQSSAVNISMQAWSSLWPKERKRQRAFFTLFGLELIVQLDIEATRTFFRTFFRLPTWMWWGFLGSSLSSFDLVLFSMYMFVLAPNSMRMSLVRHLLSDPSGAVMVRAYLER

(19) United States
(12) Patent Application Publication
(10) Pub. No.: US 2003/0220405 A1
(43) Pub. Date: Nov. 27, 2003

(54) DNA encoding an epsilon, epsilon-lycopene cyclase from romaine lettuce

(75) Inventor: Francis X. Cunningham JR., Chevy Chase, MD (US)

Correspondence Address: OBLON, SPIVAK, MCCLELLAND, MAIER & NEUSTADT, P.C., 1940 DUKE STREET, ALEXANDRIA, VA, 22314, US

(73) Assignee: University of Maryland, College Park, MD (US)

(21) Appl. No.: 10/335,846

(22) Filed: Jan. 3, 2003

Related U.S. Application Data
(63) Continuation of application No. 09/084,222, filed on May 26, 1998, now abandoned.
Application No. 09/084,222 is a continuation-in-part of application No. 08/937,155, filed on Sep. 25, 1997, now Pat. No. 6,524,811.
Application No. 08/937,155 is a division of application No. 08/624,125, filed on Mar. 29, 1996, now Pat. No. 5,744,341.

(51) Int. Cl. C12P 23/00; C07H 21/04; C12N 9/10; A01H 1/00; C12N 15/82; A61K 31/015; C12N 5/04; C12N 1/21

(52) U.S. Cl. 514/763; 800/282; 435/67; 435/69.1; 435/320.1; 435/252.33; 435/419; 435/193; 536/23.2

(57) ABSTRACT

The present invention relates to the DNA sequence for eukaryotic genes encoding ε cyclase isolated from romaine lettuce as well as vectors containing the same and hosts transformed with said vectors. The present invention provides methods for controlling the ratio of various carotenoids in a host and to the production of novel carotenoid pigments. The present invention also provides a method for treating disease by administering carotenoids obtained from transformed hosts, or by administering a composition containing the transformed hosts.

### BACKGROUND OF THE INVENTION

1. Field of the Invention

The present invention describes the DNA sequence for eukaryotic genes encoding ε lycopene cyclase as well as vectors containing the same and hosts transformed with these vectors. The present invention also provides a method for augmenting the accumulation of carotenoids and production of novel and rare carotenoids. The present invention provides methods for controlling the ratio of various carotenoids in a host. Additionally, the present invention provides a method for screening for eukaryotic genes encoding enzymes of carotenoid biosynthesis and metabolism. The invention also provides transgenic plants having therapeutic properties, methods for preparing a therapeutic composition, and methods for treating disease by administering the therapeutic plants and compositions.

2. Discussion of the Background

Carotenoid pigments with cyclic endgroups are essential components of the photosynthetic apparatus in oxygenic photosynthetic organisms (e.g., cyanobacteria, algae and plants; Goodwin, 1980). The symmetrical bicyclic yellow carotenoid pigment β-carotene (or, in rare cases, the asymmetrical bicyclic α-carotene) is intimately associated with the photosynthetic reaction centers and plays a vital role in protecting against potentially lethal photooxidative damage (Koyama, 1991). β-carotene and other carotenoids derived from it or from α-carotene also serve as light-harvesting pigments (Siefermann-Harms, 1987), are involved in the thermal dissipation of excess light energy captured by the light-harvesting antenna (Demmig-Adams & Adams, 1992), provide substrate for the biosynthesis of the plant growth regulator abscisic acid (Rock & Zeevaart, 1991; Parry & Horgan, 1991), and are precursors of vitamin A in human and animal diets (Krinsky, 1987). Plants also exploit carotenoids as coloring agents in flowers and fruits to attract pollinators and agents of seed dispersal (Goodwin, 1980). The color provided by carotenoids is also of agronomic value in a number of important crops. Carotenoids are currently harvested from plants for use as pigments in food and feed.

Two types of cyclic endgroups are commonly found in higher plant carotenoids, these are referred to as the β and ε cyclic endgroups (FIG. 2; the acyclic endgroup is referred to as the ψ or psi endgroup). These cyclic endgroups differ only in the position of the double bond in the ring. Carotenoids with two β rings are ubiquitous, and those with one β and one ε ring are common, but carotenoids with two ε rings are found in significant amounts in relatively few plants. β-Carotene (FIG. 1) has two β endgroups and is a symmetrical compound that is the precursor of a number of other important plant carotenoids such as zeaxanthin and violaxanthin (FIG. 1).

Carotenoid enzymes have previously been isolated from a variety of sources including bacteria (Armstrong et al., 1989, Mol. Gen. Genet. 216, 254-268; Misawa et al., 1990, J. Bacteriol., 172, 6704-12), fungi (Schmidhauser et al., 1990, Mol. Cell. Biol. 10, 5064-70), cyanobacteria (Chamovitz et al., 1990, Z. Naturforsch, 45c, 482-86) and higher plants (Bartley et al., Proc. Natl. Acad. Sci USA 88, 6532-36; Martinez-Ferez & Vioque, 1992, Plant Mol. Biol. 18, 981-83). Many of the isolated enzymes show a great diversity in function and inhibitory properties between sources. For example, phytoene desaturases from Synechococcus and higher plants carry out a two-step desaturation to yield ζ-carotene as a reaction product; whereas the same enzyme from Erwinia introduces four double bonds forming lycopene. Similarity of the amino acid sequences are very low for bacterial versus plant enzymes. Therefore, even with a gene in hand from one source, it is difficult to screen for a gene with similar function in another source. In particular, the sequence similarity between bacterial/fungal and cyanobacterial/plants genes is quite low.

The difficulties in isolating related genes is exemplified by recent efforts to isolated the enzyme which catalyzes the formation of β-carotene from the acyclic precursor lycopene. Although this enzyme had been isolated in a bacterium, prior to the invention described in U.S. Ser. No. 08/142,195(which is hereby incorporated by reference in its entirety),it had not been isolated from any photosynthetic organism not had the corresponding genes been identified and sequenced or the cofactor requirements established. The isolation and characterization of the enzyme catalyzing formation of β-carotene in the cyanobacterium Synechococcus PCC7942 was described by Cunningham et al. in 1993 and 1994.

The β-cyclase of Arabidopsis adds two rings to the symmetrical lycopene to form the bicyclic β-carotene, but the related ε-cyclase of Arabidopsis, which has 36% identity for the predicted amino acid sequences) adds only a single ring to form the monocyclic δ-carotene (Cunningham et al, 1996, Plant Cell 8:1613-1626; U.S. application Ser. No. 08/624,125 filed Mar. 29, 1996, which is incorporated by reference herein in its entirety). These differences in function provide a simple mechanism for adjusting the proportions of β,β-and β,ε-carotenoids while at the same time preventing formation of carotenoids with two epsilon rings.

In view of the afore-mentioned deficiencies with prior art methods of producing carotenoids with two epsilon rings, it is clear that there exists a need in the art for such methods.

### SUMMARY OF THE INVENTION

Accordingly, a first object of this invention is to provide isolated eukaryotic genes which encode enzymes which encode lycopene epsilon cyclases which form bicyclic epsilon-carotene.

A second object of the present invention is to provide vectors containing said genes.

A third object of the present invention is to provide hosts transformed with said vectors.

A further object is to provide a method for producing a lycopene epsilon cyclase using the transformed host.

A still further object is to provide the lycopene epsilon cyclase so produced.

Another object of the present invention is to provide hosts which accumulates novel or rare carotenoids or which overexpress known carotenoids.

Yet another object of the invention is to provide a method for producing novel or rare carotenoids.

Another object of this invention is to secure the expression of eukaryotic carotenoid-related genes in a recombinant prokaryotic host.

An additional object of the invention is a method of preparing a therapeutic composition comprising either the host cell which expresses the lycopene epsilon cyclase or the isolated carotenoids produced by the host cell containing the lycopene epsilon cyclase.

Another object of the invention is to provide a method for the treatment of disease by providing to a patient in need thereof, an amount of the rare carotenoids in an amount sufficient to treat the disease.

These and other objects of the present invention have been realized by the present inventors as described below.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 depicts possible routes of synthesis of cyclic carotenoids and some common plant and algal xanthophylls (oxycarotenolds) from lycopene. Activities of the ε-cyclase enzyme of lettuce are indicated by bold arrows labelled with ε. The reaction leading to ε-carotene from δ-carotene is not catalyzed by the lycopene ε cyclase of Arabidopsis (Cunningham 1996; U.S. Ser. No. 08/624,125) or other known ε-cyclases. Therefore, formation of ε-cartene and carotenoids derived from it is now made possible with the lettuce lycopene ε-cyclase describe herein. Arrows labelled with β indicate reactions synthesize by β-cyclase.

FIG. 2 depicts the carotene endgroups which are commonly found in plants.

FIG. 3 is a DNA sequence of the romaine lettuce cDNA (SEQ ID NO:1) encoding lycopene epsilon cyclase.

FIG. 4 is the predicted amino acid sequence of the romaine lettuce lycopene epsilon cyclase (SEQ ID NO:2).

FIG. 5 is a comparison between the predicted amino acid sequences of romaine lettuce (from clone DY4; SEQ ID NO:2) and Arabidopsis (from clone y2; SEQ ID NO:3) lycopene epsilon cyclase.

FIG. 6 shows the nucleotide and amino acid sequences of the ε-cyclase #3 of Adonis palaestina, which also forms bicyclic epsilon caratene.

FIG. 7 Shows a sequence comparison of the Adonis palaestina ε-cyclase #3 compared to the Adonis palaestina ε-cyclase #5, the latter of which adds only a single epsilon ring to lycopene. Five amino acid differences are noted, which may be targets for site-directed mutagenesis to form the lycopene ε-cyclase which adds two ε rings to lycopene.

### DETAILED DESCRIPTION

Romaine lettuce is one of the rare plant species that produces an abundance of a carotenoid with two epsilon rings (lactucaxahthin). The present inventors have isolated a gene encoding the epsilon cyclase from this plant, and have found that it is similar in sequence to that of Arabidopsis (about 65% identity). However, the lettuce enzyme efficiently adds two epsilon rings to lycopene to form the bicyclic epsilon-carotene.

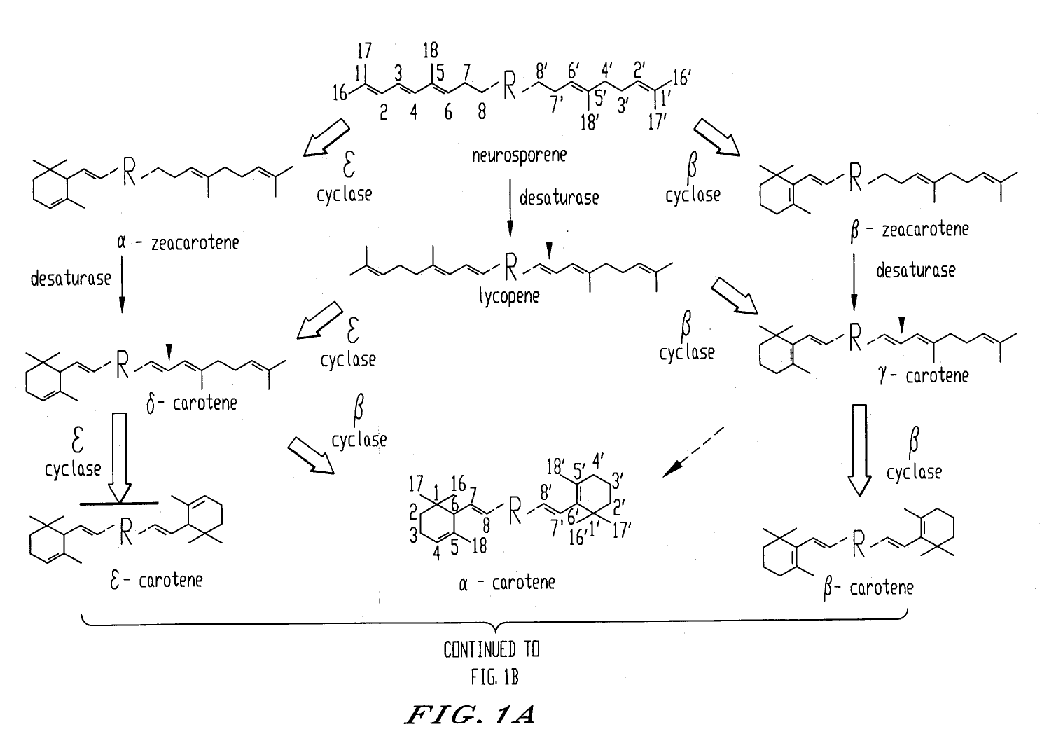

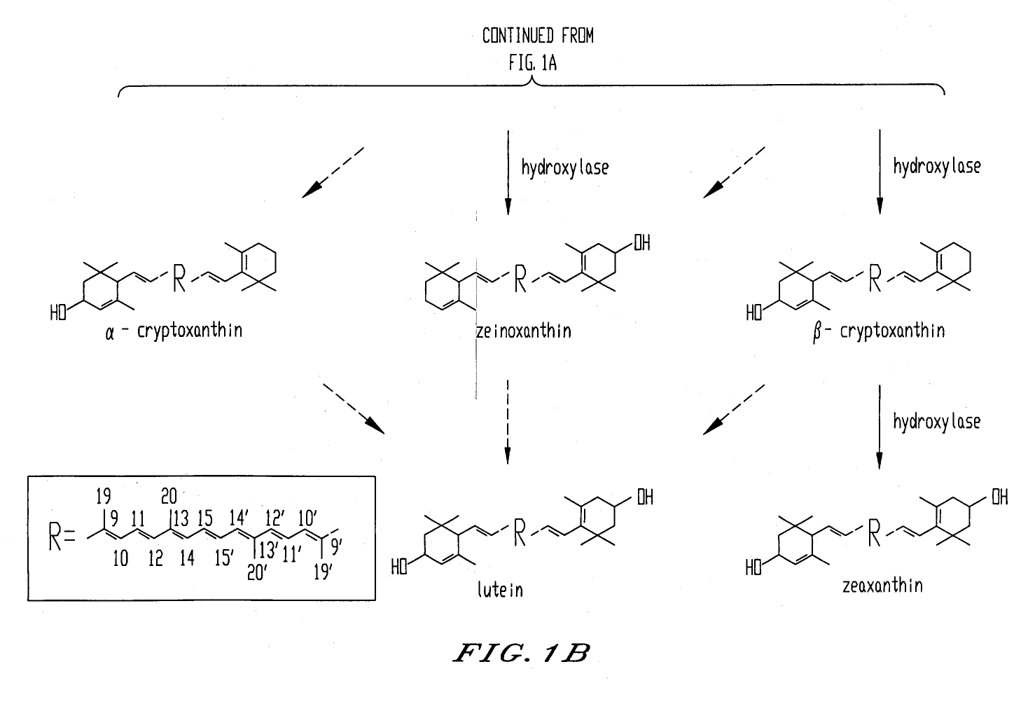

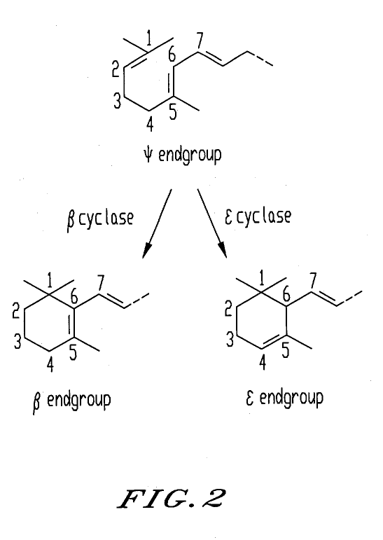

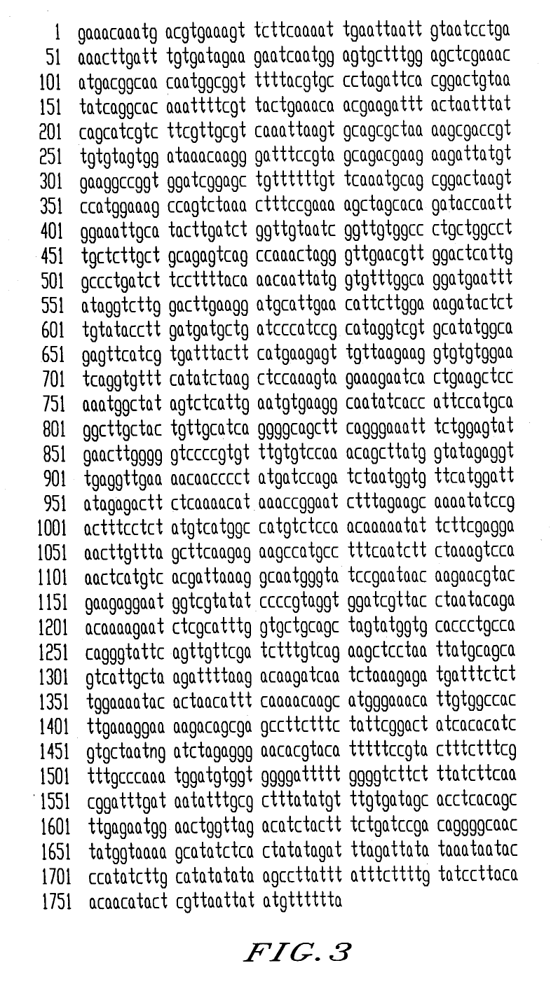

The present invention also relates to methods for transforming known carotenoids into novel or rare products. That is, currently ε-carotene (see FIG. 1) and γ-carotene can only be isolated in minor amounts. As described below, the enzymes of the invention can be produced and used to transform lycopene to bicyclic ε-carotene. With such a product in hand, bulk biosynthesis of other carotenoids derived from the bicyclic epsilon carotene are possible.

The eukaryotic genes in the carotenoid biosynthetic pathway differ from their prokaryotic counterparts in their 5′ region. As used herein, the 5′ region is the region of eukaryotic DNA which precedes the initiation codon of the counterpart gene in prokaryotic DNA. That is, when the consensus areas of eukaryotic and prokaryotic genes are aligned, the eukaryotic genes contain additional coding sequences upstream of the prokaryotic initiation codon.

The invention also relates to genes encoding lycopene epsilon cyclase which are truncated at the 5′ region of the gene. Preferably, such truncated genes are truncated to within 0-50, preferably 0-25, codons of the 5′ initiation codon of their prokaryotic counterparts as determined by alignment maps.

In addition to novel enzymes produced by truncating the 5′ region of known enzymes, novel enzymes which can participate in the formation of novel carotenoids can be formed by replacing portions of one gene with an analogous sequence from a structurally related gene. The information for adding two epsilon rings can be found in the 3′ half of the romaine lettuce gene. Thus, one example of such a hybrid gene construct would include the first half of the romaine lettuce cyclase gene in combination with the second (3′) half of another plant cyclase gene, such as the potato gene or by random of site directed mutagenesis of a mono-ε cyclase.

Vectors

The genes encoding the carotenoid enzymes as described above, when cloned into a suitable expression vector, can be used to overexpress these enzymes in a plant expression system or to inhibit the expression of these enzymes. The production or the biochemical activity of the product of epsilon-cyclase genes and cDNAs may be reduced or inhibited by a number of different approaches available to those skilled in the art [including but not limited to such methodologies or approaches as anti-sense (e.g., Gray et al (1992)Plant Mol. Biol. 19:69-87), ribozymes (e.g., Wegener et al (1994) Mol. Gen. Genet. 245:465-470), co-suppression (e.g., Fray and Grierson(1993) Plant Mol. Biol. 22:589-602), targeted disruption of the gene (e.g., Schaefer et al. (1997) Plant J. 11:1195-1206), intracellular antibodies (e.g., Rondon and Marasco (1997) Ann. Rev. Microbiol. 51:257-283 or whatever other approaches rely on the knowledge or availability of the gene, cDNA, or polypeptide and/or the sequences of these] to thereby reduce accumulation of carotenoids with psilon rings and compounds derived from them.

For example, a vector containing the gene encoding ε-cyclase can be used to increase the amount of bicyclic epsilon-carotene in an organism and thereby alter the nutritional value, pharmacology and visual appearance value of the organism. In addition, the transformed organism can be used in the formulation of therapeutic agents, for example in the treatment of cancer (Mayne et al (1996) FASEB J. 10:690-701; Tsushima et al (1995) Biol. Pharm. Bull. 18:227-233, which are both incorporated herein by reference in their entireties).

In a preferred embodiment, the vectors of the present invention contain a DNA encoding an eukaryotic IPP isomerase upstream of a DNA encoding a second eukaryotic carotenoid enzyme. The inventors have discovered that inclusion of an IPP isomerase gene increases the supply of substrate for the carotenoid pathway; thereby enhancing the production of carotenoid endproducts. This is apparent from the much deeper pigmentation in carotenoid-accumulating colonies of E. coli which also contain one of the aforementioned IPP isomerase genes when compared to colonies that lack this additional IPP isomerase gene. Similarly, a vector comprising an IPP isomerase gene can be used to enhance production of secondary metabolites of dimethylallyl pyrophosphate (such as isoprenoids, steroids, carotenoids, etc.).

Alternatively, an anti-sense strand of one of the above genes can be inserted into a vector. For example, the ε-cyclase gene can be inserted into a vector and incorporated into the genomic DNA of a host, thereby inhibiting the synthesis of ε,β carotenoids (lutein and α-carotene) and enhancing the synthesis of bicyclic epsilon carotenoids.

Suitable vectors according to the present invention comprise a eukaryotic gene encoding an enzyme involved in carotenoid biosynthesis or metabolism and a suitable promoter for the host can be constructed using techniques well known in the art (for example Sambrook et al., Molecular Cloning A Laboratory Manual, Cold Spring Harbor Laboratory, Cold Spring Harbor, N.Y., 1989).

Suitable vectors for eukaryotic expression in plants are described in Frey et al., Plant J. (1995) 8(5):693 and Misawa et al, 1994; incorporated herein by reference in their entireties.

Suitable vectors for prokaryotic expression include pACYC184, pUC119, and pBR322 (available from New England BioLabs, Bevery, Mass.), pTrcHis (Invitrogen), Bluescript SK (Stratagene) and pET28 (Novagen) and derivatives thereof.

The vectors of the present invention can additionally contain regulatory elements such as promoters, repressors selectable markers such as antibiotic resistance genes, etc.

Hosts

Host systems according to the present invention can comprise any organism that already produces carotenoids or which has been genetically modified to produce carotenoids.

Organisms which already produce carotenoids include plants, algae, some yeasts, fungi and cyanobacteria and other photosynthetic bacteria. Transformation of these hosts with vectors according to the present invention can be done using standard techniques such as those described in Misawa et al., (1990) supra; Hundle et al., (1993) supra; Hundle et al., (1991) supra; Misawa et al., (1991) supra; Sandmann et al., supra; and Schnurr et al., supra; all incorporated herein by reference in their entireties.

E. coli is an example of one type of bacteria which is suitable as a host for expression of the present enzymes (Cunningham et al, (1996) The Plant Cell 8:1613-1626, which is incorporated herein by reference in its entirety). A vector is used to construct plasmids containing genes encoding the enzymes of the invention, which vector allows it to coexist in E. coli with cloning vectors that contain the more common ColE1 origin of replication. The addition of epsilon cyclic end groups to the pink-colored lycopene will result in products that are yellow or orange-yellow in color. Therefore, the functioning of the epsilon lycopene cyclase of the invention may be detected by a change in the color of E. coli cultures that accumulate lycopene. Such assays are termed color complementation assays.

Alternatively, transgenic organisms can be constructed which include the DNA sequences of the present invention (Bird et al, 1991; Bramley et al, 1992; Misawa et al, 1994a; Misawa et al, 1994b; Cunningham et al, 1993, all of which are incorporated by reference herein in their entireties). The incorporation of these sequences can allow the controlling of carotenoid biosynthesis, content, or composition in the host cell. These transgenic systems can be constructed to incorporate sequences which allow over-expression of the carotenoid genes of the present invention. Transgenic systems can also be constructed containing antisense expression of the DNA sequences of the present invention. Such antisense expression would result in the accumulation of the substrates of the enzyme encoded by the sense strand.

Appropriate transgenic hosts include lettuce, the natural host, but also other plants such as marigold, tomato, pepper, banana, potato and the like. Essentially any plant is suitable for expressing the present enzyme, but the preferred plants are those which already produce high levels of carotenoids, and those which are normally ingested as foods or used as a source of carotenoid pigments. In particular, plants which bear fruit can be manipulated in such a way as to provide tissue-specific expression in fruit. Marigold is a particularly preferred host, because it can be used as a “bioreactor” for bulk production of carotenoids, and is actually grown commercially as a carotenoid source for chicken feed. For expression in marigold, a promoter can be used which is “flower-specific.” Another preferred transgenic plant is tomato, because this plant already produces high levels of lycopene. Indeed, it has been reported that there is a correlation between consuming tomatoes and decreased incidence of colon cancer (mayne, supra).

A Method for Screening for Eukaryotic Genes Which Encode Enzymes Involved in Carotenoid Biosynthesis

The method of the present invention comprises transforming a prokaryotic host with a DNA which may contain a eukaryotic or prokaryotic carotenoid biosynthetic gene; culturing said transformed host to obtain colonies; and screening for colonies exhibiting a different color than colonies of the untransformed host.

Suitable hosts include E. coli, cyanobacteria such as Synechococcus and Synechocystis, alga and plant cells. E. coil are preferred.

In a preferred embodiment, the above “color complementation test” can be enhanced by using mutants which are either (1) deficient in at least one carotenoid biosynthetic gene or (2) overexpress at least one carotenoid biosynthetic gene. In either case, such mutants will accumulate carotenoid precursors.

Prokaryotic and eukaryotic genomic and cDNA libraries can be screened in total for the presence of genes of carotenoid biosynthesis, metabolism and degradation. Preferred organisms to be screened include photosynthetic organisms, humans and animals.

E. coli can be transformed with these eukaryotic cDNA libraries using conventional methods such as those described in Sambrook et al, 1989 and according to protocols described by the venders of the cloning vectors.

For example, the cDNA libraries in bacteriophage vectors such as lambdaZAP (Stratagene) or lambdaZIPLOX (Gibco BRL) can be excised en masse and used to transform E. coli. Suitable vectors include pACYC184, pUC119, pBR322 (available from New England BioLabs, Bevery, Mass.). pACYC is preferred.

Transformed E. coli can be cultured using conventional techniques. The culture broth preferably contains antibiotics to select and maintain plasmids. Suitable antibiotics include penicillin, ampicillin, chloramphenicol, etc. Culturing is typically conducted at 15-45° C., preferably at room temperature (16-28° C.), for 12 hours to 7 days.

Cultures are plated and the plates are screened visually for colonies with a different color than the colonies of the host E. coli transformed with the empty vector. For example, E. coli transformed with the plasmid, pAC-BETA (described below), produce yellow colonies that accumulate β-carotene. After transformation with a cDNA library, colonies which contain a different hue than those formed by E. coli/pAC-BETA would be expected to contain enzymes which modify the structure or degree of expression of β-carotene. Similar standards can be engineered which overexpress earlier products in carotenoid biosynthesis, such as lycopene, γ-carotene, etc.

Having generally described this invention, a further understanding can be obtained by reference to certain specific examples which are provided herein for purposes of illustration only and are not intended to be limiting unless otherwise specified.

### EXAMPLE

Isolation of Lycopene Epsilon Cyclase

The lycopene epsilon cyclase was isolated from a romaine lettuce library obtained from Dr. Harry Y. Yamamoto (University of Hawaii, Honolulu) essentially as disclosed in Cunningham et al, 1996, supra, and Bugos and Yamamoto (1996) Proc. Natl. Acad. Sci. USA 93:6320-6325, both of which are incorporated herein by reference in their entireties. Functional clones were identified by the color complementation test.

Pigment Analysis

A single colony was used to inoculate 50 ml of LB containing ampicillin and chloramphenicol in a 250-ml flask. Cultures were incubated at 28° C. for 36 hours with gentle shaking, and then harvested at 5000 rpm in an SS-34 rotor. The cells were washed once with distilled H₂O and resuspended with 0.5 ml of water. The extraction procedures and HPLC were essentially as described previously (Cunningham et al, 1994).

Organisms and Growth Conditions

E. coli strains TOP10 and TOP10 F′ (obtained from Invitrogen Corporation, San Diego, Calif.) and XL1-Blue (Stratagene) were grown in Luria-Bertani (LB) medium (Sambrook et al., 1989) at 37° C. in darkness on a platform shaker at 225 cycles per min. Media components were from Difco (yeast extract and tryptone) or Sigma (NaCl). Ampicillin at 150 μg/mL and/or chloramphenicol at 50 μg/mL (both from United States Biochemical Corporation) were used, as appropriate, for selection and maintenance of plasmids.

Mass Excision and Color Complementation Screening of Romaine Lettuce cDNA Library

A cDNA library of romaine lettuce in lambda ZAPII (Bugos & Yamamoto) was obtained from Henry Yamamoto, as noted above. An aliquot of each library was treated to cause a mass excision of the cDNAs and thereby produce a phagemid library according to the instructions provided by the supplier of the cloning vector (Stratagene; E. coli strain XL1-Blue and the helper phage R408 were used). The titre of the excised phagemid was determined and the library was introduced into a lycopene-accumulating strain of E. coli TOP10 F′ by incubation of the phagemid with the E. coli cells for 15 min at 37° C. Cells had been grown overnight at 30° C. in LB medium supplemented with 2% (w/v) maltose and 10 mM MgSO₄ (final concentration), and harvested in 1.5 ml microfuge tubes at a setting of 3 on an Eppendorf microfuge (5415C) for 10 min. The pellets were resuspended in 10 mM MgSO₄ to a volume equal to one-half that of the initial culture volume. Transformants were spread on large (150 mm diameter) LB agar petri plates containing antibiotics to provide for selection of cDNA clones (ampicillin) and maintenance of pAC-LYC (chloramphenicol). Approximately 10,000 colony forming units were spread on each plate. Petri plates were incubated at room temperature for 2 to 7 days to allow maximum color development. Plates were screened visually with the aid of an illuminated 3× magnifier and a low power stage-dissecting microscope for the rare, pale pinkish-yellow to deep-yellow colonies that could be observed in the background of pink colonies. A colony color of yellow or pinkish-yellow was taken as presumptive evidence of a cyclization activity. These yellow colonies were collected with sterile toothpicks and used to inoculate 3 ml of LB medium in culture tubes with overnight growth at 37° C. and shaking at 225 cycles/min. Cultures were split into two aliquots in microfuge tubes and harvested by centrifugation at a setting of 5 in an Eppendorf 5415C microfuge. After discarding the liquid, one pellet was frozen for later purification of plasmid DNA. To the second pellet was added 1.5 ml EtOH, and the pellet was resuspended by vortex mixing, and extraction was allowed to proceed in the dark for 15-30 min with occasional remixing. Insoluble materials were pelleted by centrifugation at maximum speed for 10 min in a microfuge. Absorption spectra of the supernatant fluids were recorded from 350-550 nm with a Perkin Elmer lambda six spectrophotometer.

Analysis of Isolated Clones

Eight of the yellow colonies contained ε-carotene indicating that a single gene product catalyzes both cyclizations required to form the two ε endgroups of the symmetrical ε-carotene from the symmetrical precursor lycopene.

The availability of the romaine lettuce gene encoding the ε cyclase enables the directed manipulation of plant and algal species for modification of carotenoid content and composition. Through inactivation of the ε cyclase, whether at the gene level by deletion of the gene or by insertional inactivation or by reduction of the amount of enzyme formed (by such as antisense technology), one may increase the formation of β-carotene and other pigments derived from it. Since vitamin A is derived only from carotenoids with β endgroups, an enhancement of the production of β-carotene versus α-carotene may enhance nutritional value of crop plants. Reduction of carotenoids with ε endgroups may also be of value in modifying the color properties of crop plants and specific tissues of these plants. Alternatively, where production of α-carotene, or pigments such as lutein that are derived from α-carotene, is desirable, whether for the color properties, nutritional value or other reason, one may overexpress the ε cyclase or express it in specific tissues. Wherever agronomic value of a crop is related to pigmentation provided by carotenoid pigments the directed manipulation of expression of the ε cyclase gene and/or production of the enzyme may be of commercial value.

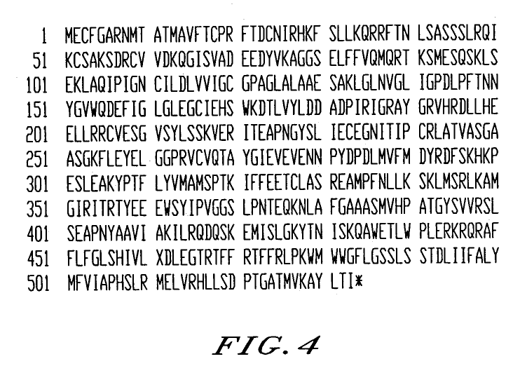

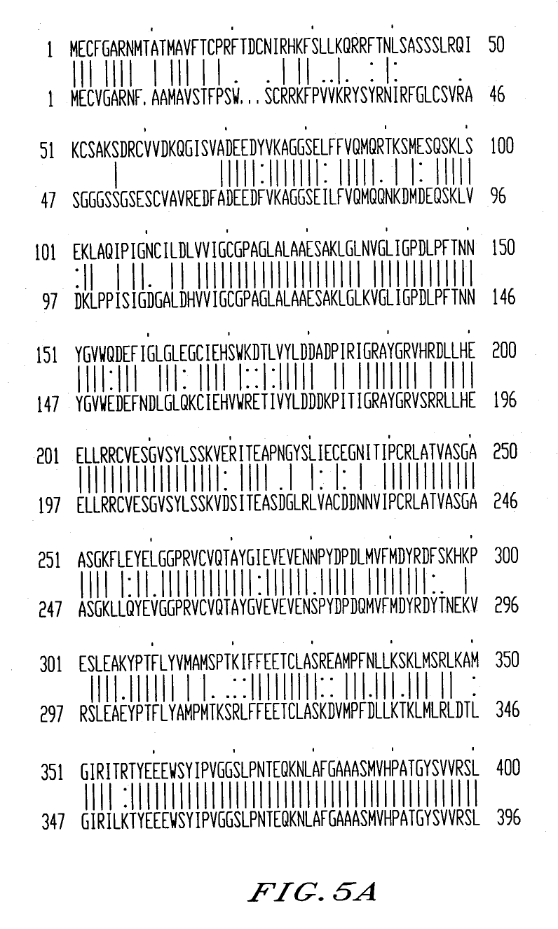

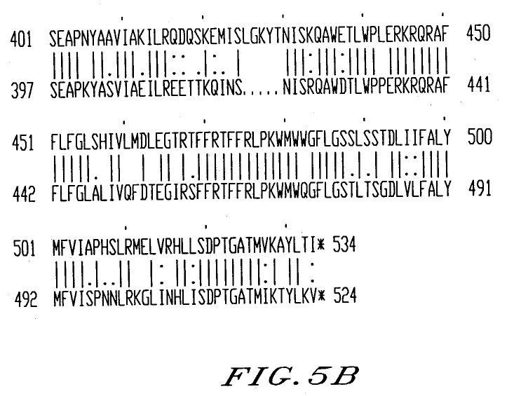

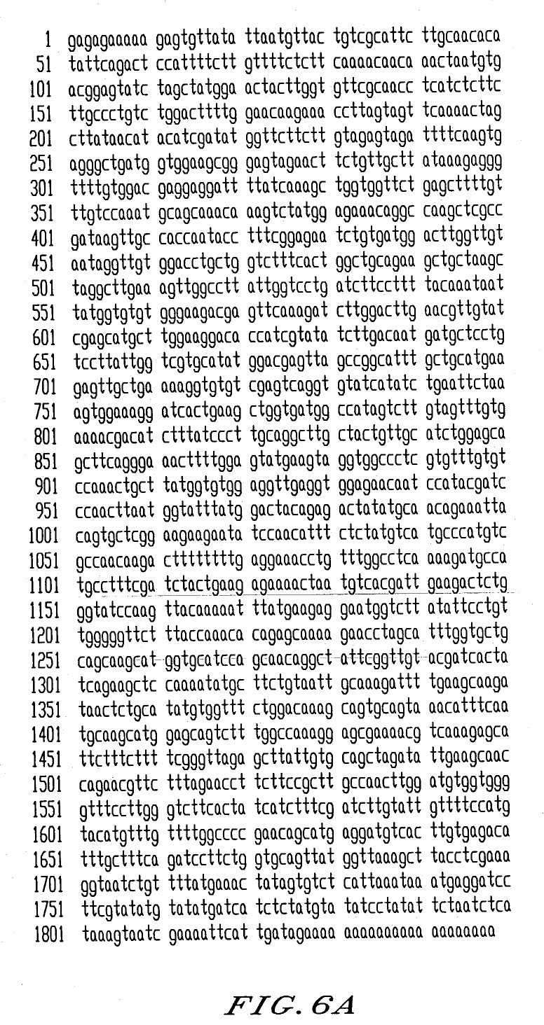

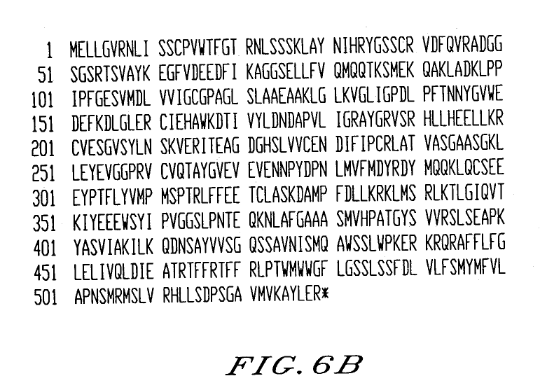

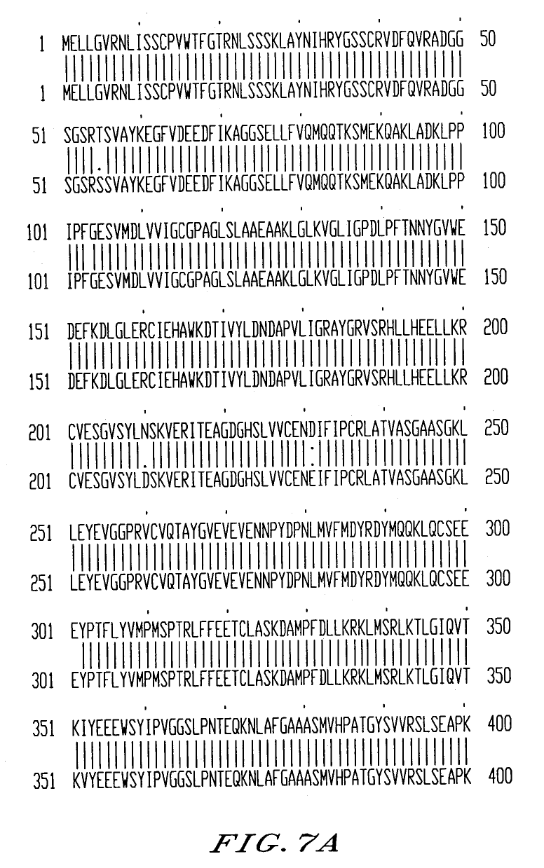

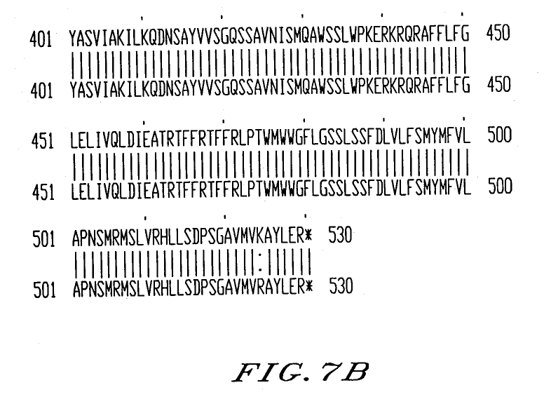

The predicted amino acid sequence of the romaine lettuce ε cyclase enzyme (SEQ ID NO:2) was determined. A comparison of the amino acid sequences of the ε cyclase enzymes of Arabidopsis thaliana and romaine lettuce (FIG. 5) as predicted by the DNA sequence of the respective genes (FIG. 3, for the ε cyclase cDNA sequence), indicates that these two enzymes have many regions of sequence similarity, but they are only about 65% identical overall at the amino acid level.

### REFERENCES

Bird et al, 1991 Biotechnology 9, 635-639.

Bishop et al., (1995) FEBS Lett. 367, 158-162.

Bramley, P. M. (1985) Adv. Lipid Res. 21, 243-279.

Bramley, P. M. (1992) Plant J. 2, 343-349.

Britton, G. (1988). Biosynthesis of carotenoids. In Plant Pigments, T. W. Goodwin, ed. (London: Academic Press), pp. 133-182.

Britton, G. (1979) Z. Naturforsch. Section C Biosci. 34, 979-985.

Britton, G. (1995) UV/Visible spectroscopy. In Carotenoids, Vol. IB: Spectroscopy, G. Britton, S. Liaaen-Jensen, H. P. Pfander, eds. (Basel: Birkhauser Verlag), pp. 13-62.

Bouvier et al., (1994) Plant J. 6, 45-54.

Cunningham et al., (1985) Photochem. Photobiol. 42: 295-307

Cunningham et al., (1993) FEBS Lett. 328, 130-138.

Cunningham et al., (1994) Plant Cell 6, 1107-1121.

Davies, B. H. (1976). Carotenoids. In Chemistry and Biochemistry of Plant Pigments, Vol. 2, T. W. Goodwin, ed (New York: Academic Press), pp. 38-165.

Del Sal et al., (1988). Nucl. Acids Res. 16, 9878.

Demmig-Adams & Adams, (1992) Ann. Rev. Plant Physiol. Mol. Biol. 43, 599-626.

Enzell & Back, (1995) Mass spectrometry. In Carotenoids, Vol. IB: Spectroscopy, G. Britton, S. Liaaen-Jensen, H. P. Pfander, eds. (Basel: Birkhauser Verlag), pp. 261-320.

Frank & Cogdell (1993) Photochemistry and function of carotenoids in photosynthesis. In Carotenoids in Photosynthesis. A. Young and G. Britton, eds. (London: Chapman and Hall). pp. 253-326.

Goodwin, T. W. (1980). The Biochemistry of the Carotenoids. 2nd ed, Vol. 1 (London: Chapman and Hall.

Horvath et al., (1972) Phytochem. 11, 183-187.

Hugueney et al., (1995) Plant J. 8, 417-424.

Hundle et al., (1991) Photochem. Photobiol. 54, 89-93.

Jensen & Jensen, (1971) Methods Enzymol. 23, 586-602.

Kargl & Quackenbush, (1960) Archives Biochem. Biophys. 88, 59-63.

Kargl et al., (1960) Proc. Am. Hort. Soc. 75, 574-578.

Kieber et al., (1993) Cell 72, 427-441.

Koyama, Y. (1991) J. Photochem. Photobiol., B, 9, 265-80.

Krinsky, N. I. (1987) Medical uses of carotenoids. In Carotenoids, N. I. Krinsky, M. M. Mathews-Roth, and R. F. Taylor, eds. (New York: Plenum), pp. 195-206.

Kyte & Doolittle, (1982) J. Mol. Biol. 157, 105-132.

LaRossa & Schloss, (1984) J. Biol. Chem. 259, 8753-8757.

Misawa et al., (1994a) Plant J. 6, 481-489.

Misawa et al., (1994b) J. Biochem, Tokyo, 116, 980-985.

Norris et al., (1995) Plant Cell 7, 2139-2149.

Pecker et al., (1996) Submitted to Plant Mol. Biol.

Perry et al., (1986) J. Bacteriol. 168, 607-612.

Persson & Argos, (1994) J. Mol. Biol. 237, 182-192.

Plumley & Schmidt, (1987) Proc. Nat. Acad. Sci. USA 83, 146-150.

Plumley & Schmidt, (1995) Plant Cell 7, 689-704.

Rossmann et al., (1974) Nature 250, 194-199.

Rock & Zeevaart (1991) Proc. Nat. Acad. Sci. USA 88, 7496-7499.

Rost et al., (1995) Protein Science 4, 521-533.

Sambrook et al., (1989) Molecular Cloning: A Laboratory Manual, 2nd edition (Cold Spring Harbor, N.Y.: Cold Spring Harbor Laboratory Press).

Sancar, A. (1994) Biochemistry 33, 2-9.

Sander & Schneider, (1991) Proteins 9, 56-68.

Sandmann, G. (1994) Eur. J. Biochem. 223, 7-24.

Scolnik & Bartley, (1995) Plant Physiol. 108, 1342.

Siefermann-Harms, D. (1987) Physiol. Plant. 69, 561-568.

Spurgeon & Porter, (1980). Biosynthesis of carotenoids. In Biochemistry of Isoprenoid Compounds, J. W. Porter, and S. L. Spurgeon, eds. (New York: Wiley), pp. 1-122.

Tomes, M. L. (1963) Bot. Gaz. 124, 180-185.

Tomes, M. L. (1967) Genetics 56, 227-232.

Tuveson et al., (1986) J. Bacteriol. 170, 4675-4680.

Van Beeumen et al., (1991) J. Biol. Chem. 266, 12921-12931.

Weedon & Moss, (1995) Structure and Nomenclature. In Carotenoids, Vol. IB: Spectroscopy, G. Britton, S. Liaaen-Jensen, H. P. Pfander, eds. (Basel: Birkhauser Verlag), pp. 27-70.

Wierenga et al., (1986) J. Mol. Biol. 187, 101-107.

Zechmeister, L. (1962) Cis-Trans Isomeric Carotenoids, Vitamins A and Arylpolyenes. Springer-Verlag, Vienna.

Having now fully described the invention, it will be apparent to one of ordinary skill in the art that many changes and modifications can be made thereto without departing from the spirit or scope of the invention as set forth herein.

What is claimed as new and is desired to be secured by Letters Patent of the United States is:

1. An isolated eukaryotic enzyme which converts lycopene to epsilon, epsilon-carotene.

2. An isolated eukaryotic enzyme of claim 1 having the amino acid sequence of SEQ ID NO: 2.

3. An isolated DNA sequence comprising a gene encoding the eukaryotic ε cyclase of claim 2.

4. The isolated DNA sequence according to claim 3, having the nucleic acid sequence of SEQ ID NO: 1.

5. An expression vector comprising the DNA sequence of claim 3.

6. A host containing the expression vector of claim 5.

7. The host of claim 6, wherein said host is E. coli.

8. The host of claim 6, wherein said host is a plant.

9. The host of claim 8, wherein said host is marigold.

10. The host of claim 8, wherein said host is tomato.

11. A composition comprising the host of claim 6.

12. A composition comprising the host of claim 8.

13. A composition comprising bicyclic epsilon carotene obtained from the host of claim 6.

14. A composition comprising bicyclic epsilon carotene obtained from the host of claim 8.

15. A method for treating disease comprising administering to a patient in need thereof, an amount of the composition of claim 13 sufficient to treat said disease.

16. A method for treating disease comprising administering to a patient in need thereof, an amount of the composition of claim 14 sufficient to treat said disease.
